(19) United States
(12) Patent Application Publication
(10) Pub. No.: US 2023/0263560 A1
(43) Pub. Date: Aug. 24, 2023

(54) Flexible Intramedullary Device Tether

(71) Applicant: Texas Scottish Rite Hospital for Children, Dallas, TX (US)

(72) Inventors: William Arthur Pierce, Dallas, TX (US); Philip L. Wilson, Pilot Point, TX (US)

(21) Appl. No.: 18/006,300

(22) PCT Filed: Jul. 20, 2021
(86) PCT No.: PCT/US2021/042366

§ 371 Date: Jan. 20, 2023

Related U.S. Application Data
(60) Provisional application No. 63/054,124, filed on Jul. 20, 2020.

(51) Int. Cl. A61B 17/82; A61B 17/72

(52) U.S. Cl. CPC A61B 17/82; A61B 17/7233; A61B 2017/564

(57) ABSTRACT

A tether for an implantable bone device, the tether including a retention portion for holding the implantable bone device in place, wherein the retention portion comprises a recess configured to receive a protruding proximate end of the device when the device is positioned inside a bone, wherein the recess is open at a first end and closed at a second end; and a fastening portion for affixing the tether to the bone, wherein the fastening portion comprises one or more through fastener openings, wherein each through fastener opening is configured to receive a bone fastener, wherein the fastening portion is adjustable, and wherein the fastening portion is shaped to be less thick that a height of the retention portion.

### CROSS-REFERENCE TO RELATED APPLICATIONS

This PCT International Application claims priority to U.S. Provisional Application No. 63/054,124 filed on Jul. 20, 2020, the contents of which are incorporated by reference herein.

### STATEMENT OF FEDERALLY FUNDED RESEARCH

None.

### TECHNICAL FIELD OF THE INVENTION

The present invention relates in general to the field of devices for fixation to bone. In particular, the present invention relates to implantable, adjustable devices for fixation to bone.

### BACKGROUND OF THE INVENTION

U.S. Patent Application Publication No. 2018/0317982 A1, by Giordano, et al., is said to disclose an implant for coupling an exterior surface of a bone to an intramedullary nail, comprising a body having a bone securement portion and a nail securement portion; said bone securement portion including at least one through hole adapted and configured to receive therein a bone fastener, the through hole having a first axis; and said nail securement portion defining a receptacle having an opened end and a closed end both aligned along a second axis, the receptacle being adapted and configured for receiving therein a portion of an intramedullary nail; wherein the first axis and the second axis are adapted and configured such that the opened end of the receptacle is oriented toward the interior of a long bone when the nail securement portion is fastened to the exterior surface of the long bone.

It is desirable to have a tether for holding an intramedullary nail or any intramedullary device inserted into a medullary cavity in place when installed in a bone, where the tether is small, implantable, and flexible for adjustment to the bone's contour. It is also desirable to have a targeting device and a clamp for use with such a intramedullary device.

### SUMMARY OF THE INVENTION

In some embodiments of the disclosure, a tether for an implantable bone device is disclosed as including a retention portion for holding the implantable bone device in place, wherein the retention portion includes a recess configured to receive a proximate end of the implantable bone device when the implantable bone device is positioned inside a bone with the proximate end protruding from the bone, wherein the recess is open at a first end and closed at a second end; and a fastening portion for affixing the tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the retention portion. In one aspect, the implantable bone device is an intramedullary rod, an intramedullary nail, an inter-locking screw, a Küntscher nail, a percutaneous screw, or a percutaneous pin. In one aspect, the tether includes a bone-side contour to approximate a contour of the bone to which it is to be affixed. In another aspect, the fastening portion is adjustable to create a bone-side contour to approximate a contour of the bone to which it is to be affixed. In another aspect, the tether includes a smooth outside contour to reduce irritation of tissue when implanted. In another aspect, the fastening portion includes a first tab disposed on a first side of the retention portion having a first through fastener opening and a second tab disposed on a second side of the retention portion having a second through fastener opening. In another aspect, the fastening portion includes a single tab on one side of the retention portion having a single through fastener opening. In another aspect, the tether further includes one or more bone-side spikes protruding from a bone-side surface of the tether and configured to penetrate the bone when the tether is affixed to the bone.

In some embodiments of the disclosure, a tether kit is disclosed as including a tether for an implantable bone device, the tether including a retention portion for holding the implantable bone device in place, wherein the retention portion includes a recess configured to receive a proximate end of the implantable bone device when the implantable bone device is positioned inside a bone with the proximate end protruding from the bone, wherein the recess is open at a first end and closed at a second end; and a fastening portion for affixing the tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the nail portion. In one aspect, the tether includes a bone-side contour to approximate a contour of the bone to which it is to be affixed. In another aspect, the fastening portion is adjustable to create a bone-side contour to approximate a contour of the bone to which it is to be affixed. In another aspect, the tether includes a smooth outside contour to reduce irritation of tissue when implanted. In another aspect, the fastening portion includes a first tab disposed on a first side of the nail portion having a first through fastener opening and a second tab disposed on a second side of the nail portion having a second through fastener opening. In another aspect, the fastening portion includes a single tab on one side of the nail portion having a single through fastener opening. In another aspect, the tether kit further includes one or more implantable bone devices. In another aspect, the tether further includes one or more bone-side spikes protruding from a bone-side surface of the tether and configured to penetrate the bone when the tether is affixed to the bone.

In some embodiments of the disclosure, a method for using a tether for an implantable bone device is disclosed as including providing a patient requiring installation of the implantable bone device and the tether; installing the implantable bone device in a bone with a proximate end of the implantable bone device exposed; positioning the tether on the proximate end of the implantable bone device; and affixing the tether to the bone to hold the implantable bone device in place; wherein the tether includes a retention portion for holding the implantable bone device in place, wherein the retention portion includes a recess configured to receive a proximate end of the implantable bone device when the implantable bone device is positioned inside the bone with the proximate end protruding from the bone, wherein the recess is open at a first end and closed at a second end; and a fastening portion for affixing the tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the retention portion. In one aspect, the tether includes a bone-side contour to approximate a contour of the bone to which it is to be affixed. In another aspect, the bone portion is adjustable to create a bone-side contour to approximate a contour of the bone to which it is to be affixed. In another aspect, the tether includes a smooth outside contour to reduce irritation of tissue when implanted. In another aspect, the fastening portion includes a first tab disposed on a first side of the nail portion having a first through fastener opening and a second tab disposed on a second side of the nail portion having a second through fastener opening. In another aspect, the fastening portion includes a single tab on one side of the nail portion having a single through fastener opening. In another aspect, the tether further includes one or more bone-side spikes protruding from a bone-side surface of the tether and configured to penetrate the bone when the tether is affixed to the bone.

In some embodiments of the disclosure, a computer-readable medium is disclosed as storing computer-readable instructions which, when acted upon by a 3D printer, cause the 3D printer to print a tether for an implantable bone device, the tether including a retention portion for holding the implantable bone device in place, wherein the retention portion includes a recess configured to receive a proximate end of the implantable bone device when the implantable bone device is positioned inside a bone with the proximate end protruding from the bone, wherein the recess is open at a first end and closed at a second end; and a fastening portion for affixing the implantable bone device tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the retention portion.

In some embodiments of the disclosure, a targeting device for a bone drill or an implantable bone device is disclosed as including a handle; a sleeve attached to or integral with the handle at a proximal end of the sleeve, including at least a trough with a smoothly-curved cross-section and configured to receive a bone drill bit or the implantable bone device lengthwise; and at least one first spike at the distal end of the trough, configured to engage bone tissue to hold the distal end of the sleeve in place during use. In one aspect, the sleeve further includes a partial tube with a smoothly-curved cross-section, configured to be attached lengthwise to the trough to form a tube. In another aspect, the sleeve and the trough are configured to be held together by at least one of one or more o-rings; one or more clamps; one or more tongue and groove joints, one or more keys and slots, or one or more articles of shrink tubing. In another aspect, the at least one first spike includes two spikes formed on opposite sides of the distal end of the sleeve; the at least one first spike includes two spikes formed proximal to each other on a same side of the distal end of the sleeve; or the at least one first spike includes a single flat spike formed along the circumference of the distal end of the sleeve. In another aspect, the sleeve or the partial tube includes at least one second spike at the distal end of the sleeve or the partial tube. In another aspect, targeting device further includes a mallet anvil disposed at the proximal end of the sleeve and configured to receive taps from a hammer at least to aid the at least one first spike to engage the bone tissue.

In some embodiments of the disclosure, a method of using a targeting device for drilling bone is disclosed as including providing a patient in need of an implantable bone device installation through a hole drilled in a bone; providing the targeting device including: a handle; a sleeve attached to or integral with the handle at a proximal end of the sleeve, including at least a trough with a smoothly-curved cross-section and configured to receive a bone drill bit or the implantable bone device lengthwise; and at least one first spike at the distal end of the trough, configured to engage bone tissue to hold the distal end of the sleeve in place during use; making an incision over a drilling site; positioning the targeting device proximal to the drilling site, wherein the at least one first spike engages the bone proximal to the drilling site; aligning the tube to point to the drilling site; inserting a drill bit into the tube; and drilling the hole in the bone. In one aspect, the method further includes removing the partial tube from the trough; inserting the implantable bone device into at least a portion of the trough; and inserting the implantable bone device into the hole.

In some embodiments of the disclosure, a method of using a targeting device for inserting an implantable bone device is disclosed as including providing a patient in need of an implantable bone device installation through a hole drilled in a bone; providing the targeting device including: a handle; a sleeve attached to or integral with the handle at a proximal end of the sleeve, including at least a trough with a smoothly-curved cross-section and configured to receive a bone drill bit or the implantable bone device lengthwise; and at least one first spike at the distal end of the trough, configured to engage bone tissue to hold the distal end of the sleeve in place during use; inserting the targeting device into an incision over a hole drilled in a bone; positioning the targeting device proximal to the hole, wherein the at least one first spike engages the bone proximal to the hole; aligning the trough, without the partial tube attached, to point to the hole; inserting the implantable bone device into at least a portion of the trough; and inserting the implantable bone device into the hole.

In some embodiments of the disclosure, a clamp for manipulating tether for an implantable bone device is disclosed as including two clamp arms joined at their proximal ends or between their proximal and distal ends and configured such that their distal ends can be brought together, wherein the distal ends of the two clamp arms are configured to grip the tether; wherein the tether includes a retention portion for holding the implantable bone device in place, wherein the retention portion includes a recess configured to receive a proximate end of the implantable bone device when the implantable bone device is positioned inside a bone with the proximate end protruding from the bone, wherein the recess is open at a first end and closed at a second end; and a fastening portion for affixing the tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the retention portion. In one aspect, the two arms are joined rotationally and wherein one or both arms are capable of rotating to bring their distal ends together. In another aspect, the two arms are joined at their proximal ends and wherein one or both arms are capable of flexing to bring their distal ends together. In another aspect, the distal ends of the two arms are configured to grip the fastening portion of the tether. In another aspect, the distal ends of the two arms each include a skin retractor configured to retract skin around an incision from the tether when the distal ends of the two arms grip the tether.

In some embodiments of the disclosure, a keyless chuck is disclosed as including a receptacle configured to receive a proximal end of the implantable bone device or an implantable bone device tamp, wherein no key is required to secure the implantable bone device or the implantable bone device tamp in the receptacle, and wherein the receptacle is configured to rotate the implantable bone device or the implantable bone device tamp when the keyless chuck is rotated. In one aspect, the keyless chuck further includes an impact anvil surface. In another aspect, the keyless chuck further includes an implantable bone device tamp, wherein the tamp is detachably engageable with the receptacle.

In some embodiments of the disclosure, a kit is disclosed as including at least one tether for an implantable bone device, each tether including: a retention portion for holding an implantable bone device in place, wherein the retention portion includes a recess configured to receive a proximate end of the implantable bone device when the implantable bone device is positioned inside a bone with the proximate end protruding from the bone, wherein the recess is open at a first end and closed at a second end; and a fastening portion for affixing the tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the retention portion; at least one targeting device including: a handle; a sleeve attached to or integral with the handle at a proximal end of the sleeve, including at least a trough with a smoothly-curved cross-section and configured to receive a bone drill bit or the implantable bone device lengthwise; and at least one first spike at the distal end of the trough, configured to engage bone tissue to hold the distal end of the sleeve in place during use; at least one clamp for manipulating the tether, each at least one clamp including: two clamp arms joined at their proximal ends or between their proximal and distal ends and configured such that their distal ends can be brought together, wherein the distal ends of the two clamp arms are configured to grip the tether; and at least one keyless chuck, each keyless chuck including a receptacle configured to receive a proximal end of the implantable bone device or an implantable bone device tamp, wherein no key is required to secure the implantable bone device or the implantable bone device tamp in the receptacle, and wherein the receptacle is configured to rotate the implantable bone device or the implantable bone device tamp when the keyless chuck is rotated.

### BRIEF DESCRIPTION OF THE DRAWINGS

For a more complete understanding of the features and advantages of the present invention, reference is now made to the detailed description of the invention along with the accompanying figures, in which:

FIG. 1 shows an embodiment of an intramedullary device tether.

FIGS. 2A and 2B show various views of another embodiment of the intramedullary device tether. FIGS. 2C and 2D are photographs of the embodiment shown in FIGS. 2A and 2B.

FIGS. 3A-3C are photographs of another embodiment of the intramedullary device tether. FIGS. 3D and 3E are photographs of the embodiment of FIGS. 3A-3C with an insertion tool for use with any embodiment of the intramedullary device tether.

FIGS. 4A-4C show various views of another embodiment of the intramedullary device tether.

FIGS. 5A and 5B show the embodiment of the intramedullary device tether of FIGS. 3A-3E with bone-side spikes that can be a feature of any embodiment of the intramedullary device tether.

FIGS. 5C and 5D show an embodiment of tether of the present invention with three exemplary bone-side spikes and one exemplary through fastener opening.

FIG. 6 is a flow chart that shows a method embodiment for using the intramedullary device tether.

FIG. 7A shows an embodiment of the targeting device of the present invention.

FIG. 7B shows another embodiment of the targeting device of the present invention.

FIG. 7C shows another embodiment of the targeting device of the present invention.

FIG. 7D shows another view of the embodiment of the targeting device of FIG. 7A with a partial tube attached.

FIG. 7E shows another embodiment of the targeting device of the present invention.

FIG. 7F shows another embodiment of the targeting device of the present invention.

FIG. 7G shows another view of the embodiment of the targeting device of FIG. 7E.

FIG. 7H shows another embodiment of the targeting device of the present invention.

FIG. 7I shows another view of the embodiment of the targeting device of FIG. 7H.

FIG. 7J shows another embodiment of the targeting device of the present invention.

FIG. 7K shows another view of the targeting device of FIG. 7J.

FIG. 7L shows the one or more spikes of the targeting device in the form of a “fish mouth.”

FIG. 7M shows the one or more spikes of the targeting device in the form of a “snake tongue.”

FIG. 7N shows the one or more spikes of the targeting device in the form of a “tooth.”

FIG. 8 is a flow chart that shows a method for using the targeting device for drilling bone.

FIG. 9 is a flow chart that shows a method for using the targeting device for inserting an intramedullary device.

FIG. 10A shows a clamp for manipulating the intramedullary device tether.

FIG. 10B shows a clamp of the present invention engaging a tethering device of the present invention.

FIG. 10C shows another view of a clamp of the present invention engaging a tethering device of the present invention.

FIG. 11A shows an embodiment of a keyless chuck of the present invention.

FIG. 11B shows an embodiment of an implantable bone device tamp of the present invention to be used in conjunction with the keyless chuck.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

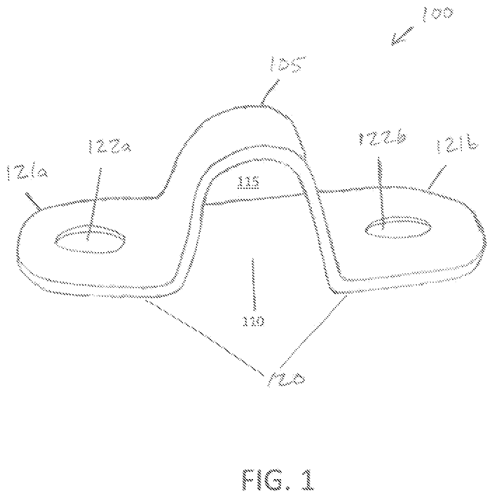

FIG. 1 shows an exemplary embodiment of the present invention, an intramedullary device tether 100 including a retention portion 105 for holding in place an intramedullary nail or any implantable bone device (not shown) inserted into a medullary cavity. The present invention is usable with any implantable bone device, e.g., an intramedullary rod, an intramedullary nail, an inter-locking nail, a Küntscher nail, a percutaneous screw, or a percutaneous pin. Any reference herein to an intramedually device is not limiting and is exemplary of any implantable bone device. Any reference herein to an intramedullary device tether is not limiting and is exemplary of a tether that can be used with any implantable bone device. The retention portion 105 includes a recess 110 configured to receive a proximate end of the intramedullary device when the intramedullary device is positioned inside a bone (not shown) with the proximate end protruding from the bone, wherein the recess is open at a first end and closed at a second end by a surface 115 (e.g., to firmly retain the proximate end of the intramedullary device); and a fastening portion 120 for affixing the intramedullary device tether 100 to the bone, wherein the fastening portion comprises one or more through fastener openings, and wherein the fastening portion is shaped to be less thick than a height of the retention portion 105. The one or more fastener openings may have threads. In the intramedullary device tether 100, the fastening portion 120 comprises a first tab 121a disposed on a first side of the retention portion 105 having a first through fastener opening 122a and a second tab 121b disposed on a second side of the retention portion 105 having a second through fastener opening 122b, wherein the first tab 121a and the second tab 121b are shaped to be less thick than the height of the retention portion 105.

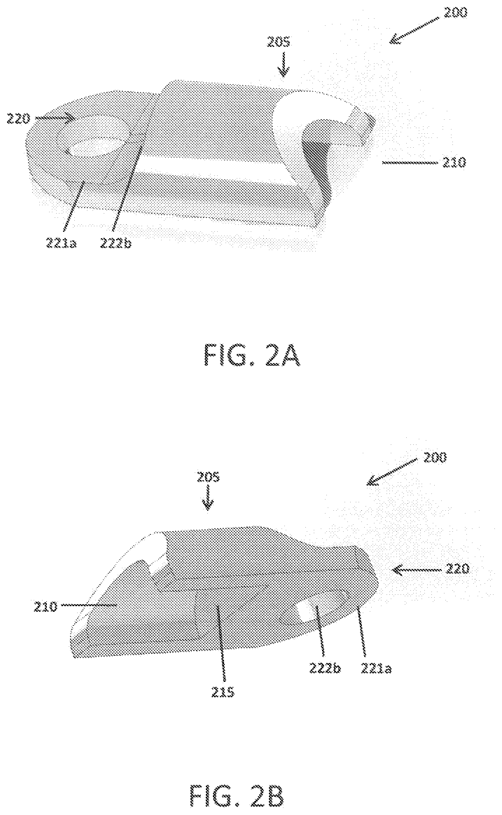

FIGS. 2A and 2B show various views of another embodiment of the present invention, the intramedullary device tether 200. A retention portion 205 includes a recess 210 to receive a proximate end of the intramedullary device when the intramedullary device (not shown) is positioned inside a bone with the proximate end protruding from the bone, wherein the recess is open at a first end and closed at a second end by a surface 215 (e.g., to firmly retain the proximate end of the intramedullary device); and a fastening portion 220 for affixing the intramedullary device tether 200 to the bone, wherein the fastening portion comprises a single through fastener opening, and wherein the fastening portion is shaped to be less thick than a height of the retention portion 205. In the intramedullary device tether 200, the fastening portion 220 comprises a tab 221a disposed at an end of the retention portion 205 opposite the opening for the intramedullary device and a through fastener opening 221b, wherein the tab 221a is shaped to be less thick that the height of the retention portion 205. FIGS. 2C and 2D are photos of the intramedullary device tether 200 fastened to an artificial bone and tethering an intramedullary device 225 (see FIG. 2D).

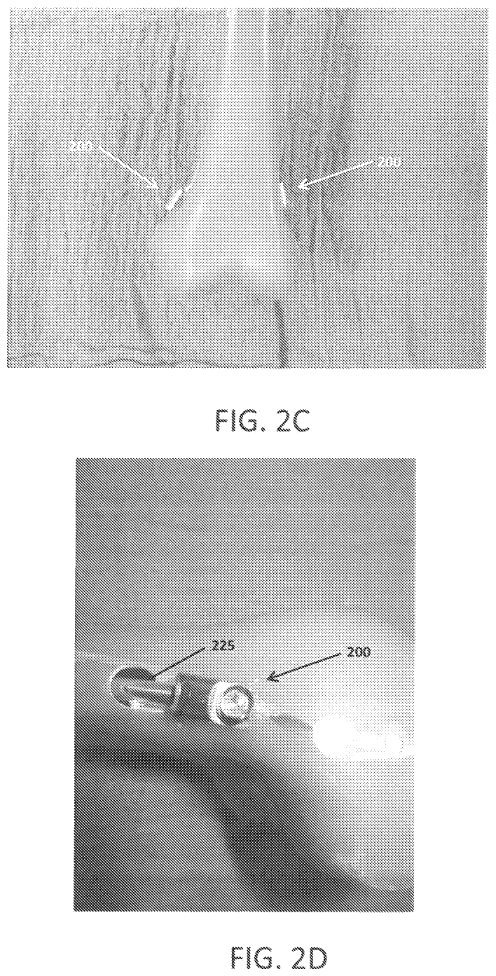

FIGS. 3A-3C are photos showing various views of another embodiment of the present invention, the intramedullary device tether 300 with a recess 310 with an interior diameter and length sufficient to accommodate any intramedullary device with a proximate end with an outer diameter of up to that interior diameter. The intramedullary device tether 300 is also shown with capture wings 330a and 330b on each side of the retention portion 305 to reduce or eliminate lateral movement of the intramedullary device tether 300. The two capture wings 330a and 330b shown are non-limiting examples of one or more such capture wings that may be disposed on one or both sides or ends of retention portions of embodiments of the present invention. FIGS. 3D and 3E are photos showing the intramedullary device 300 with an insertion tool 335, which may be used with any embodiment of the present invention.

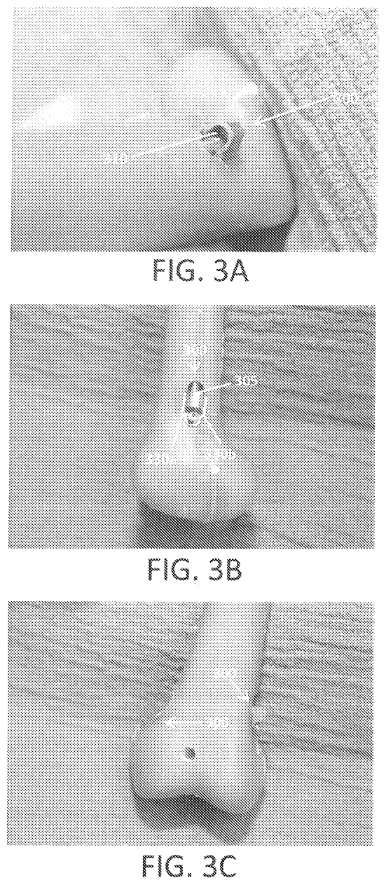

FIGS. 4A-4C show various views of another embodiment of the present invention, the the intramedullary device tether 400. A retention portion 405 includes a recess 410 to receive a proximate end of the intramedullary device (not shown) when the intramedullary device is positioned inside a bone with the proximate end protruding from the bone, wherein the recess is open at a first end and closed at a second end (e.g., to firmly retain the proximate end of the intramedullary device); and a fastening portion 420 for affixing the intramedullary device tether 400 to the bone, wherein the fastening portion comprises a single through fastener opening, and wherein the fastening portion is shaped to be less thick than a height of the retention portion 405. In the intramedullary device tether 400, the recess 410 is contoured to approximate or to match the shape of the proximate end of the intramedullary device to more securely tether the device. The outer surface 415 of the retention portion 405 may be contoured to follow the contour of the recess 410 at least in part to reduce irritation of soft tissue when implanted, e.g., when the incision in soft tissue made to install the intramedullary device and the intramedullary device tether is closed over the device and the tether after the installation. In the intramedullary device tether 400, the fastening portion 420 comprises a tab 421a disposed at an end of the retention portion 405 opposite the opening for the intramedullary device and a through fastener opening 422a, wherein the tab 421a is shaped to be less thick than the height of the retention portion 405.

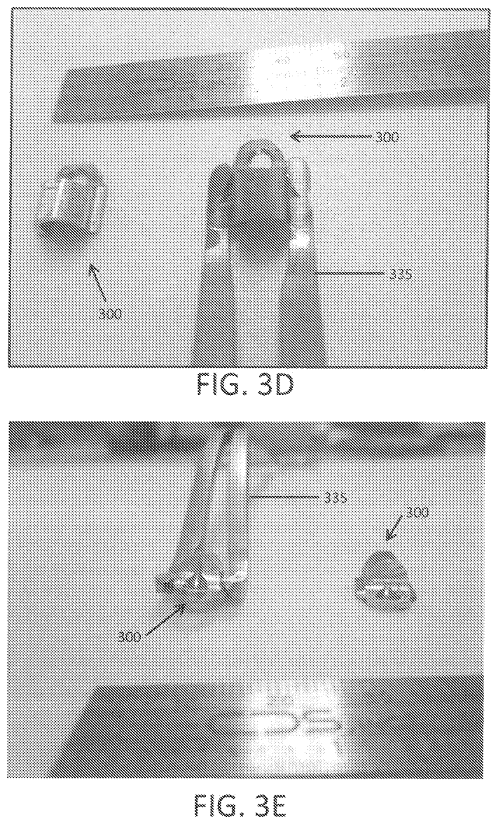

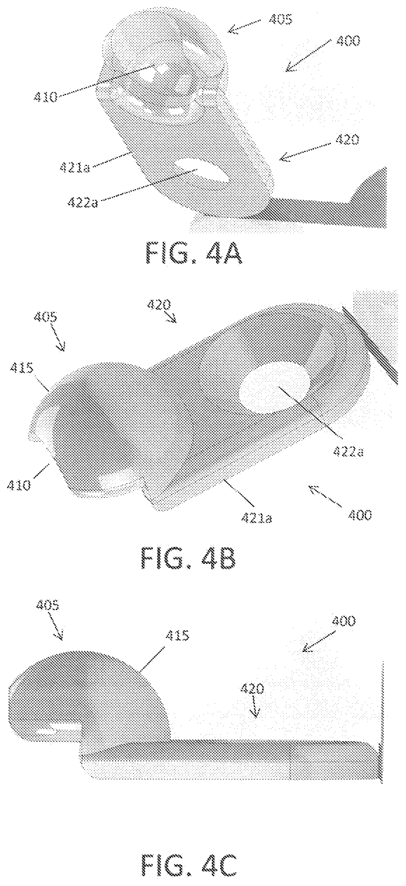

FIGS. 5A and 5B show the embodiment of the intramedullary device tether 300 of FIG. 3A-3E with bone-side spikes 505a and 505b, which can be a feature of any embodiment of the present invention. The two bone-side spikes 505a and 505b shown are non-limiting examples of one or more such spikes that may be disposed on the bone-side surface of embodiments of the present invention to reduce or eliminate lateral movement of the intramedullary device tether or rotation of the intramedullary device tether. The bone-side spikes 505a and 505b are configured to penetrate a bone when the intramedullary device tether is affixed to the bone.

FIGS. 5C and 5D show an embodiment of the tether 300 with three exemplary, non-limiting bone-side spikes 505a, 505b, and 505c and one exemplary through fastener opening 510a. Embodiments of the present invention are not limited to these numbers of bone-side spikes and through fastener openings. Embodiments of the present invention include tethers with one, two, three or more bone-side spikes or one, two, three, or more through fastener openings, or any combination.

An embodiment of the present invention may include a bone-side contour to approximate a contour of the bone to which it is to be affixed. An embodiment of the present invention may include a fastening portion that is adjustable to create a bone-side contour to approximate a curved contour or a flat contour of the bone to which it is to be affixed, e.g. by bending at least a portion of the fastening portion, e.g., a tab. An embodiment of the present invention may include a smooth outside contour to reduce irritation of soft tissue when implanted, e.g., when the incision that is made in soft tissue to install the intramedullary device and the intramedullary device tether is closed over the device and the tether after the installation.

A fastening portion or a component thereof may be substantially perpendicular to a recess in an embodiment, as shown in FIGS. 1, 2A-2D, 3A-3E, 4A-4C, and 5A-5D-, or at an angle to the recess, e.g., an angle of 0, 5, 10, 20, 30, 40, 45, 50, 60, 70, 80, 85, or 89 degrees.

One or more embodiments of the present invention may be included in a kit that may also include one or more items generally used in conjunction with implanting an intramedullary device tether, such as one or more bone fasteners, one or more intramedullary devices, one or more tether holders, or one or more insertion tools.

Any embodiment of the intramedullary device tether, including tethers 100, 200, 300, and 400, may be made using 3D printing or additive manufacturing, and computer-readable instructions for operating a 3D printer to print such an intramedullary device tether may be stored on or in a computer-readable storage medium. Such a 3D-printed tether may include any of the features that may be included in a non-3D-printed tether. One exemplary advantage of a 3D-printed intramedullary device tether is that data from imagery of the bone to which the tether is to be affixed, such as data from a CT scan of the bone, may be used to contour the bone-side surface of the tether to match the contour of the bone with high precision.

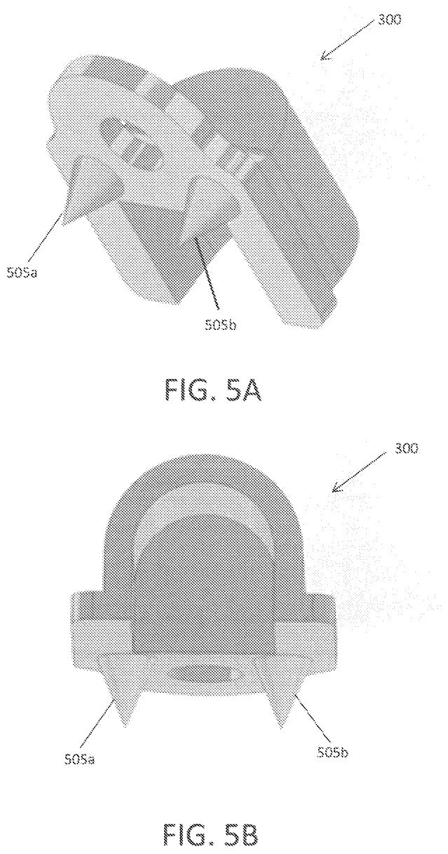

FIG. 6 is a flowchart of a method embodiment 600 for using an intramedullary device tether that includes, in block 605, providing a patient requiring installation of an intramedullary device and an intramedullary device tether. Block 610 includes installing the intramedullary device in a bone with a proximate end of the intramedullary device exposed. Included in block 615 is positioning the intramedullary device tether on the proximate end of the intramedullary device. Block 620 includes fastening the intramedullary device tether to the bone to hold the intramedullary device in place. Block 625 includes the intramedullary device tether including a retention portion for holding an intramedullary device in place, the retention portion including a recess configured to receive a proximate end of the intramedullary device when the intramedullary device is positioned inside a bone with the proximate end protruding from the bone, the recess being open at a first end and closed at a second end; and a fastening portion for affixing the intramedullary device tether to the bone, wherein the fastening portion comprises one or more through fastener openings, wherein each through fastener opening is configured to receive a bone fastener, and wherein the fastening portion is shaped to be less thick than a height of the retention portion.

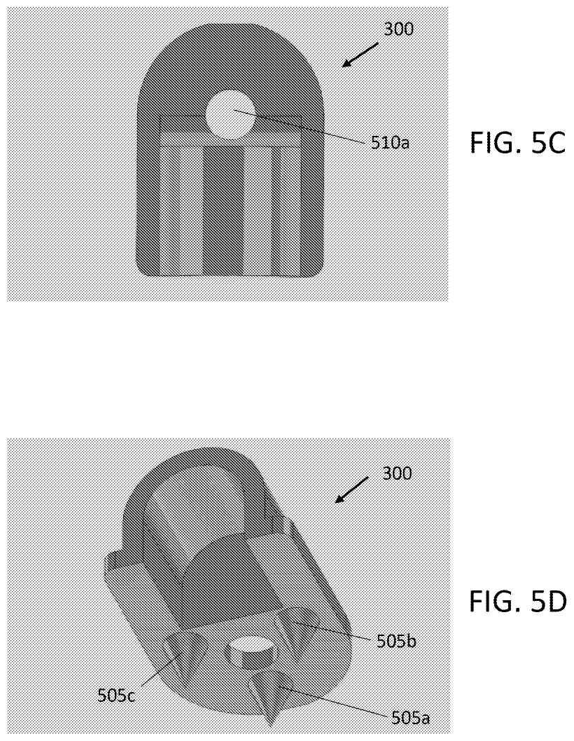

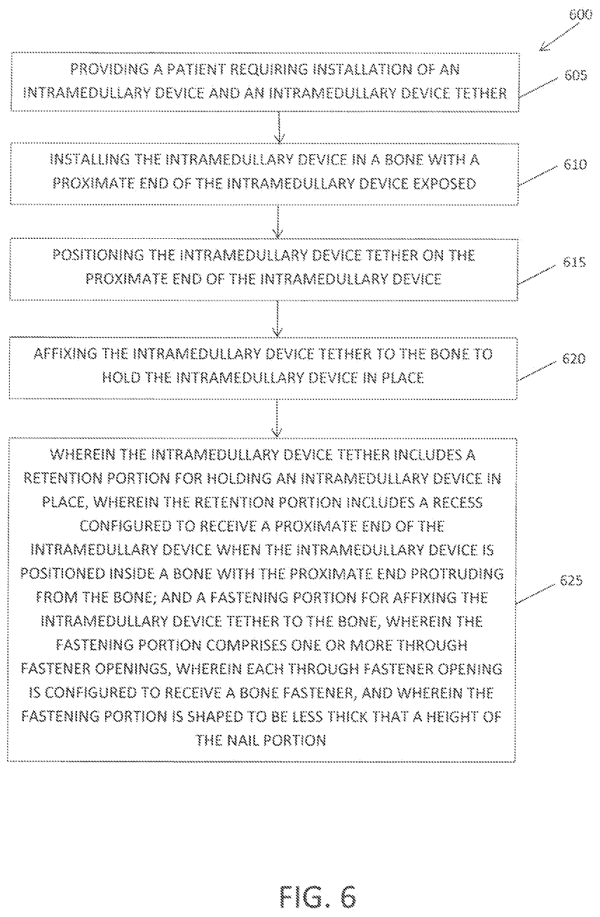

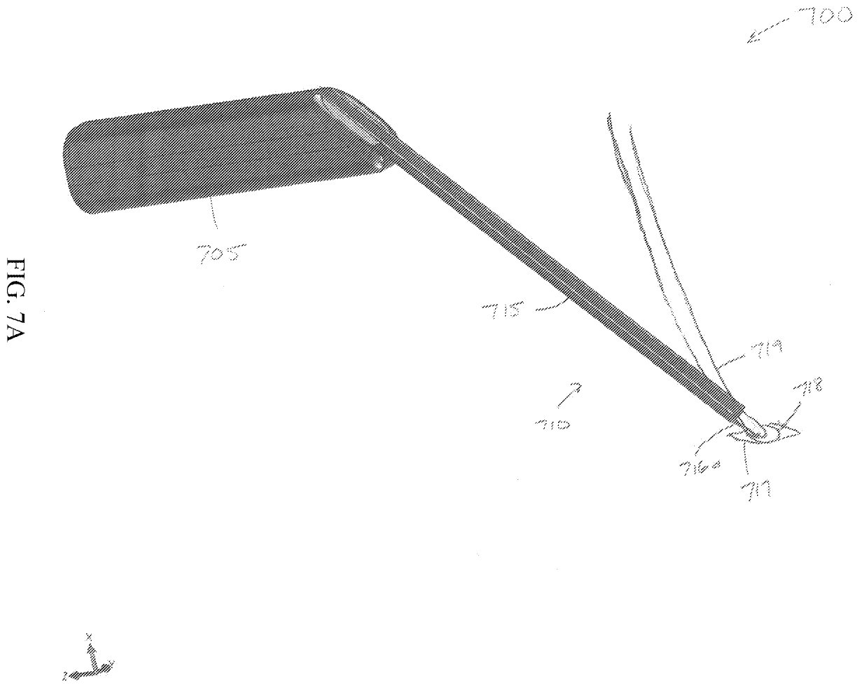

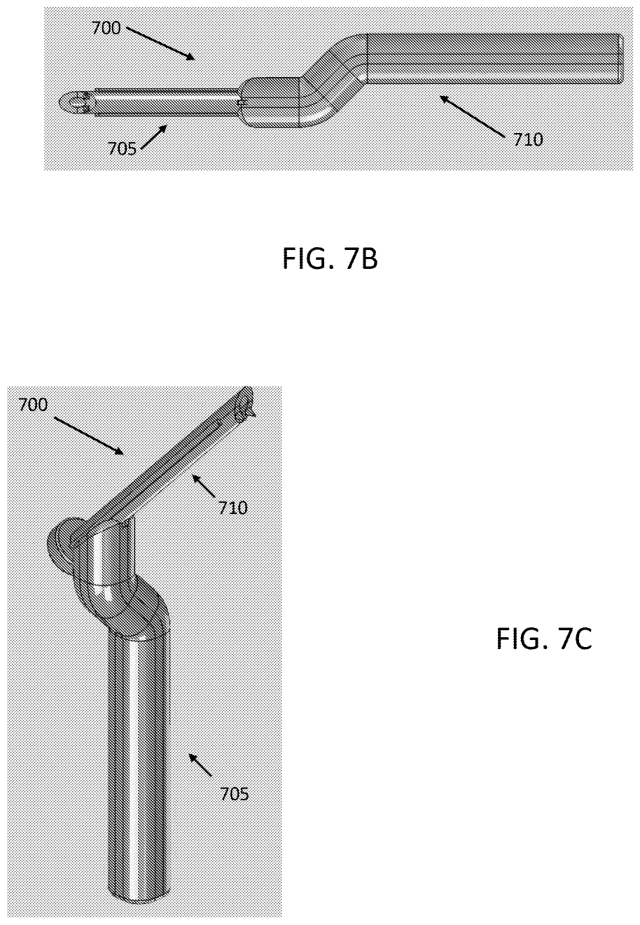

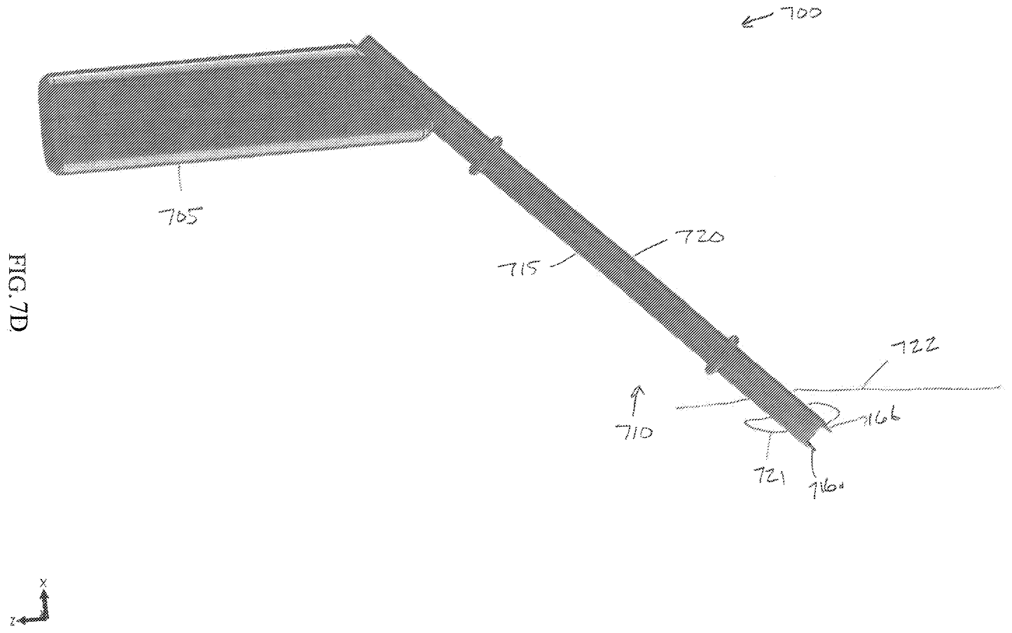

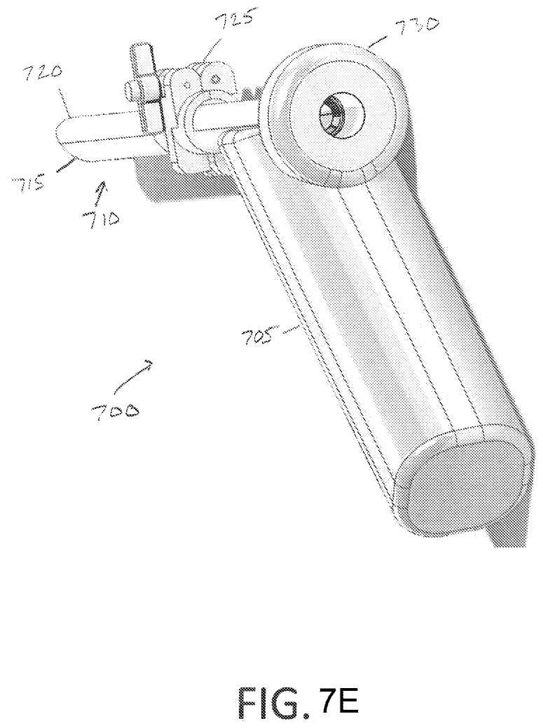

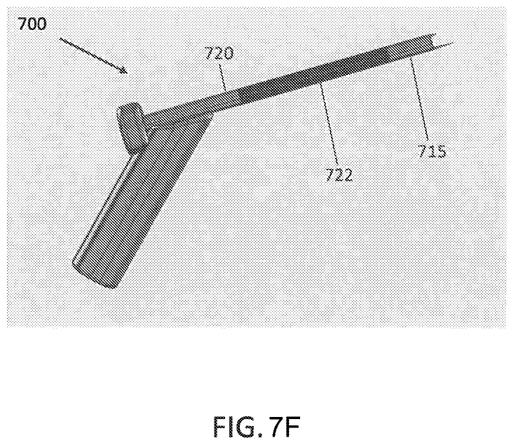

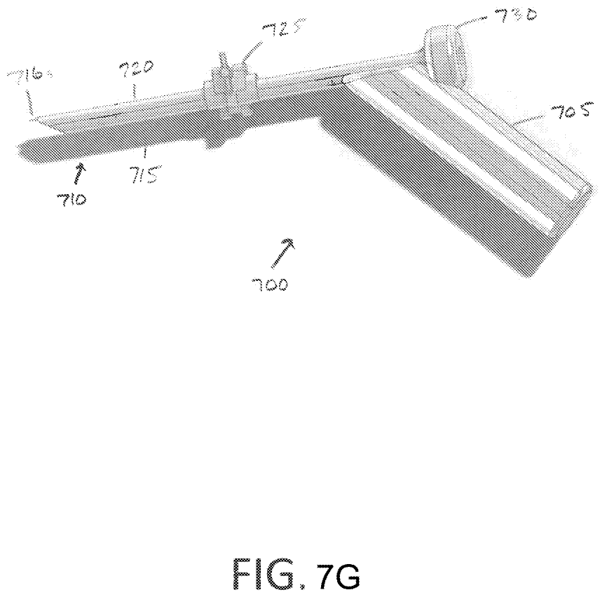

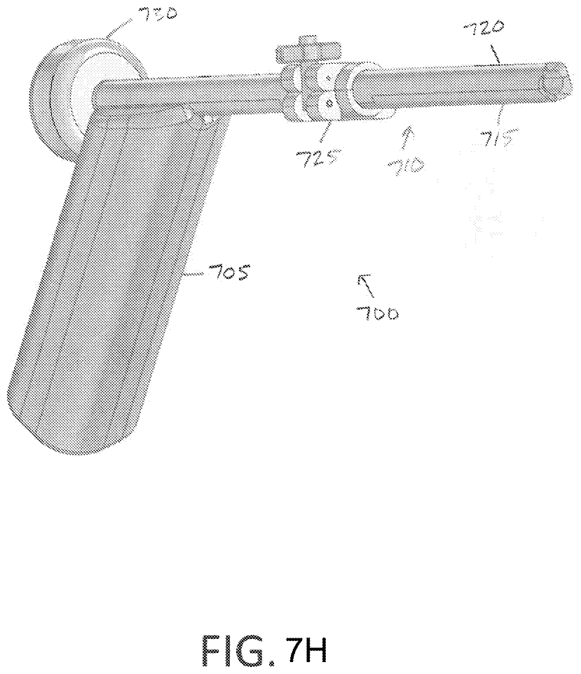

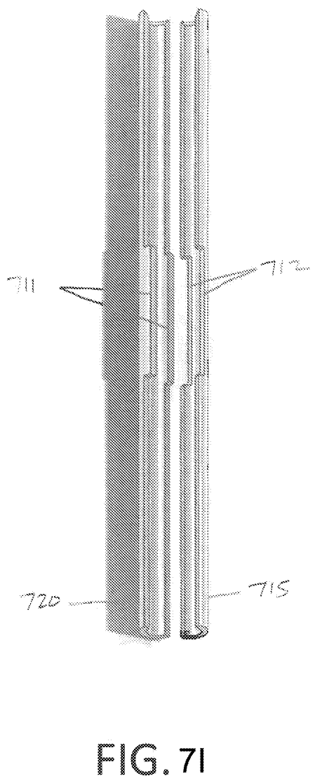

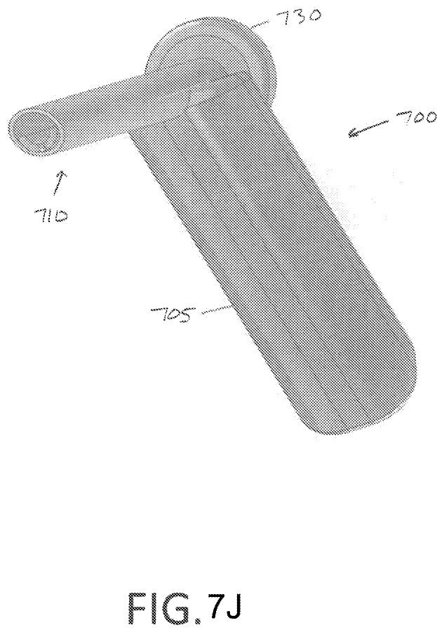

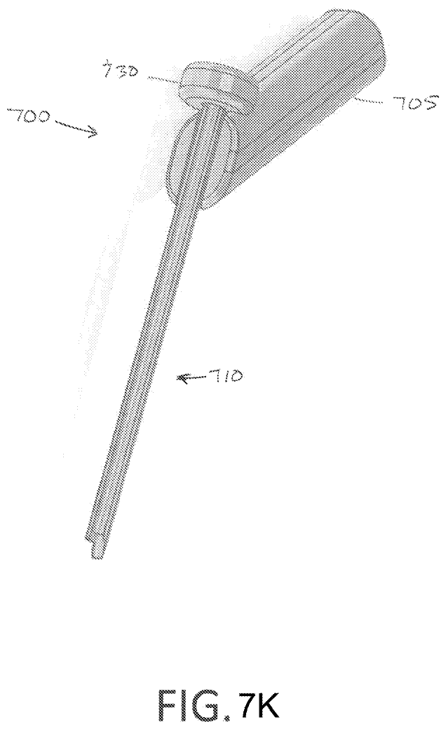

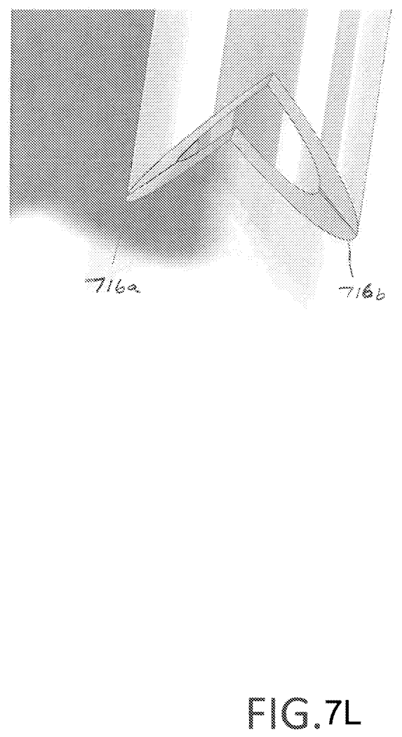

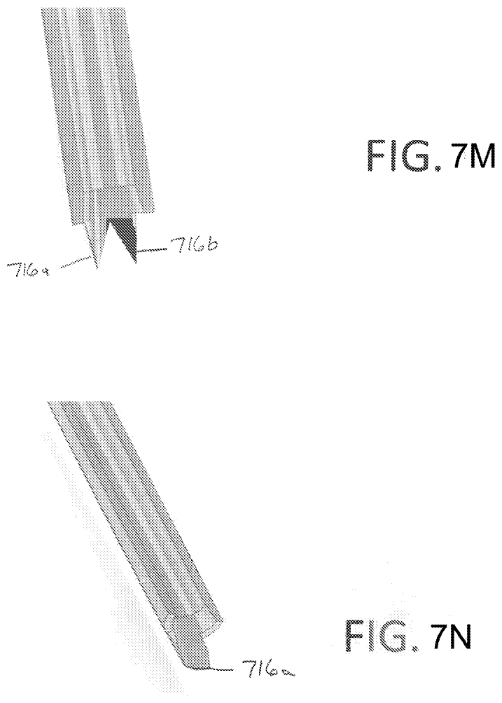

FIG. 7A shows an embodiment of a targeting device 700 of the present invention that can be used to assist bone drilling or intramedullary device insertion. The targeting device 700 includes a handle 705 and a sleeve 710 that is attached to or integral with the handle 705 at or near a proximal end of the sleeve 710 relative to a user of the targeting device 700. The sleeve 710 may be situated on the handle 705 centered on the plane that bisects the handle 705 side-to-side as shown in FIG. 7A or offset from that plane. The sleeve 710 comprises at least a trough 715 and has a smoothly-curved cross-section to minimize irritating or damaging interaction with a patient's soft tissues during bone drilling or intramedullary device insertion. The sleeve 710 also has at least one first spike 716a at the distal end of the trough 715, configured to engage bone tissue to hold the distal end of the trough 715 in place during use of the targeting device 700. The targeting device 700 is shown with the first spike 716 engaging a bone 717 in which a hole 718 has been drilled, and with an intramedullary device 719 positioned in a portion of the trough 715, ready for insertion into the hole 718.

As shown in FIG. 7B, the sleeve 710 of the targeting device 700 may be offset from the plane that bisects the handle 705 side-to-side, and the sleeve 710 may be at an angle to the plane that bisects the handle 705 side-to-side. In FIG. 7C, the sleeve 710 of the targeting device 700 is shown both offset from the bisecting plane and at an angle to the bisecting plane of the handle 705.

FIG. 7D shows another view of the embodiment of the targeting device 700 with an additional component of the sleeve 710, a partial tube 720 that is configured to be attached lengthwise to the trough 715 to form a tube and to be detached from the trough 715. The trough 715 and the partial tube 720 are held together mechanically by e.g.: one or more o-rings, one or more sleeve clamps; one or more articles of shrink tubing, which may be sterilizable; or one or more tongue and groove joints, wherein each tongue and groove joint may be configured as a square, triangular, round, or other geometrically shaped tongue and groove joint. The partial tube 720 has a smoothly-curved cross-section to minimize irritating or damaging interaction with a patient's soft tissues during bone drilling or intramedullary device insertion. First spike 716a and second spike 716b are shown at the distal ends of the trough 715 and the partial tube 720, respectively. The targeting device 700 is shown with the tube formed by the trough 715 and the partial tube 720 inserted into an incision 721 in soft tissue 722, ready for a drill bit (not shown) to be inserted into the tube for drilling a hole in a bone, or ready for an intramedullary device to be inserted into the tube for insertion into a bone.

FIG. 7E shows another embodiment of the targeting device 700 of the present invention that can be used to assist bone drilling or intramedullary device insertion. The targeting device 700 includes a handle 705, a sleeve 710 that is attached to or integral with the handle 705 at or near a proximal end of the sleeve 710 relative to a user of the targeting device 700, with a partial tube 720 held to the trough 715 with the sleeve clamp 725. The sleeve 710 comprises at least a trough 715 and has a smoothly-curved cross-section to minimize irritating or damaging interaction with a patient's soft tissues during bone drilling or intramedullary device insertion. The sleeve 710 also has at least one first spike 716a at the distal end of the trough 715, configured to engage bone tissue to hold the distal end of the trough 715 in place during use of the targeting device 700. The targeting device 700 also has a mallet anvil 730 which may be tapped with a hammer at least to aid engagement of the bone tissue by the at least one first spike 717a.

FIG. 7F shows another embodiment of the targeting device 700 in which the partial tube 720 is held to the trough 715 by an article of shrink tubing 722.

FIG. 7G shows another view of the embodiment of the targeting device 700 of FIG. 7E. The targeting device 700 includes the handle 705, the sleeve 710, with the partial tube 720 held to the trough 715 with the sleeve clamp 725, and the mallet anvil 730. The sleeve 710 is shown with the first spike 716a at the distal end of the trough 715, which distal end is beveled in this embodiment.

FIG. 7H shows another embodiment of the targeting device 700 of the present invention. The targeting device 700 includes the handle 705, the sleeve 710, with the partial tube 720 held to the trough 715 with the sleeve clamp 725, and the mallet anvil 730.

FIG. 7I shows the keyed sleeve 710 of FIG. 7H with keys 711 and slots 712.

FIG. 7J shows another embodiment of the targeting device 700 of the present invention. The targeting device 700 includes the handle 705, the sleeve 710, and the mallet anvil 730.

FIG. 7K shows another view of the targeting device 700 of FIG. 7J with the outer sleeve removed.

FIG. 7L shows two spikes 716a and 716b of the targeting device 700 (not shown in its entirety) formed on opposite sides of the distal end of the sleeve (“fish mouth”). FIG. 7M shows two spikes 716a and 716b of the targeting device 700 (not shown in its entirety) formed adjacent to the each other on a same side of the distal end of the sleeve (“snake tongue”). FIG. 7N shows a spike 716a of the targeting device 700 (not shown in its entirety) formed as a single flat spike formed along the circumference of the distal end of the sleeve (“tooth”).

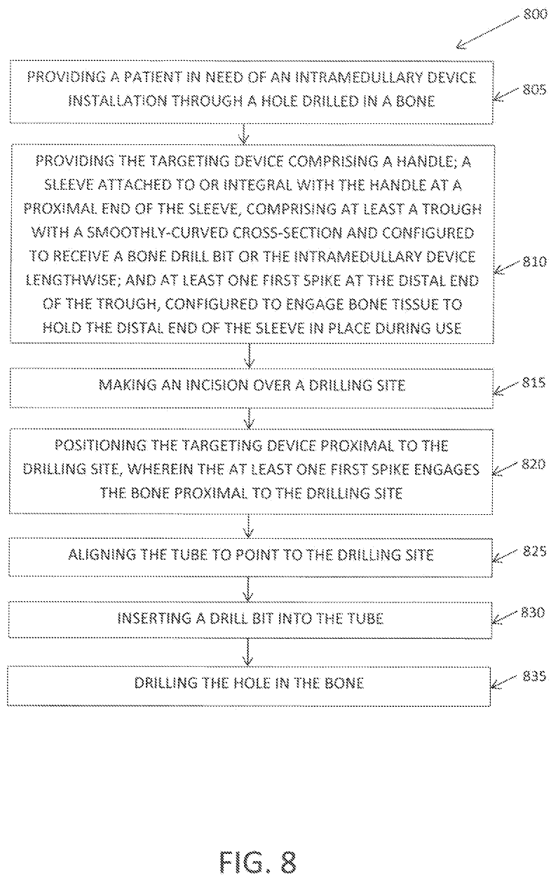

FIG. 8 is a flowchart of a method embodiment 800 for using the targeting device 700 for drilling bone. Method 800 includes block 805, providing a patient in need of an implantable bone device installation through a hole drilled in a bone. Method 800 further includes block 810, providing the targeting device 700 as described herein. Block 815 includes making an incision over a drilling site. Positioning the targeting device proximal to the drilling site, wherein the at least one first spike engages the bone proximal to the drilling site is included in block 820. Block 825 includes aligning the tube to point to the drilling site. Block 830 includes inserting a drill bit into the tube. Block 835 includes drilling the hole in the bone.

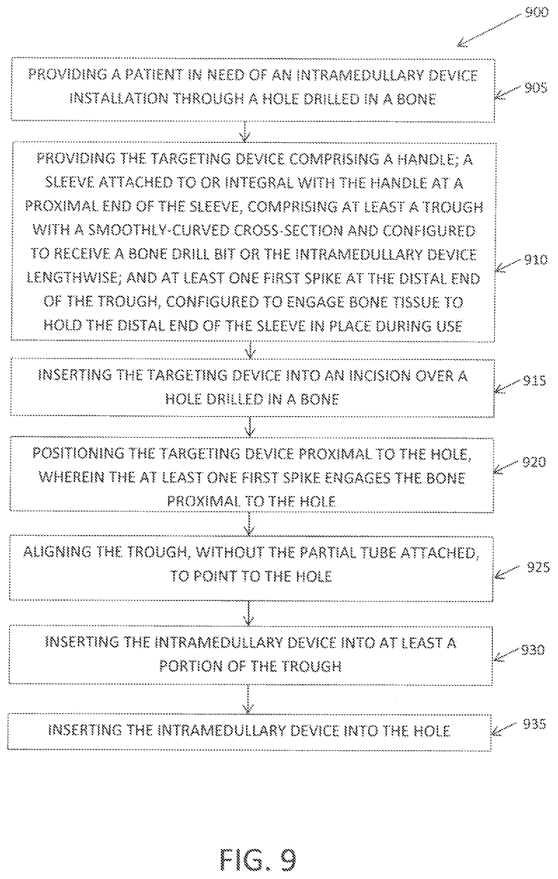

FIG. 9 is a flowchart of a method embodiment 900 for using the targeting device 700 for inserting an implantable bone device. Method 900 includes block 905, providing a patient in need of an implantable bone device installation through a hole drilled in a bone. Block 910 includes providing the targeting device 700 described herein. Block 915 includes inserting the targeting device into an incision over a hole drilled in a bone. Positioning the targeting device proximal to the hole, wherein the at least one first spike engages the bone proximal to the hole is included in block 920. Block 925 includes aligning the trough, without the partial tube attached, to point to the hole. Block 930 includes inserting the implantable bone device into at least a portion of the trough. Block 935 includes inserting the implantable bone device into the hole.

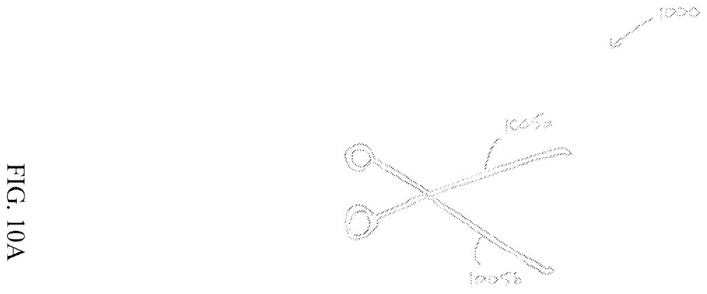

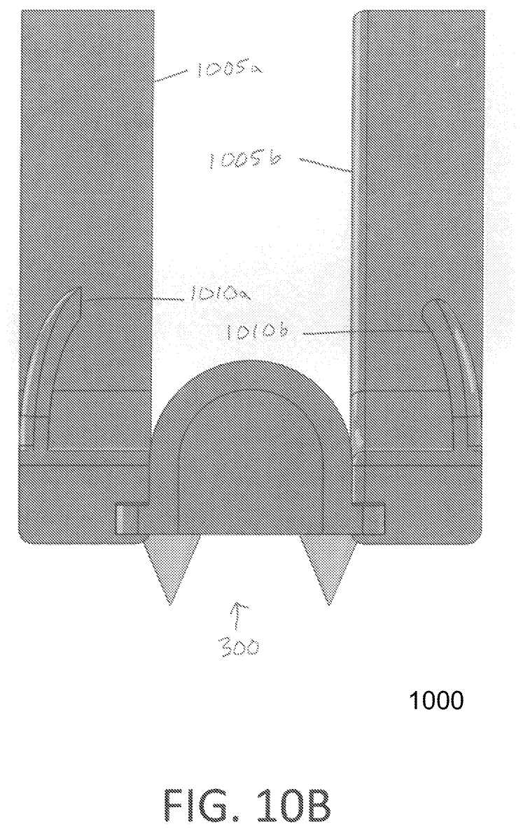

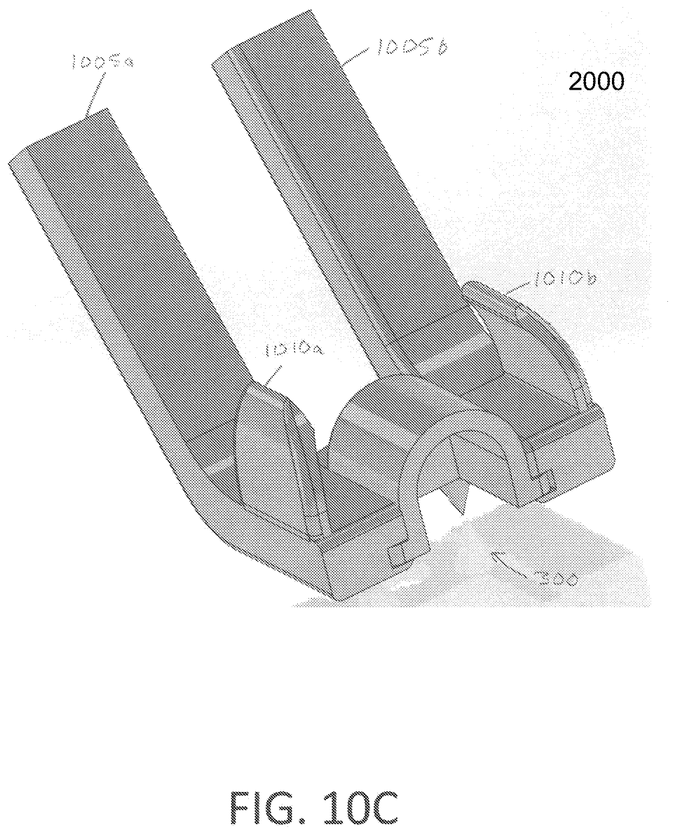

FIG. 10A shows a clamp 1000 of the present invention for manipulating a tether of the present invention. The clamp 1000 includes two clamp arms 1005a and 1005b joined at their proximal ends, relative to a user, or between their proximal and distal ends, and configured such that their distal ends can be brought together, wherein the distal ends of the two clamp arms 1005a and 1005b are configured to grip any of the intramedullary device tethers described herein (not shown).

FIG. 10B shows a clamp 1000 of the present invention engaging a tether of the present invention. The two clamp arms 1005a and 1005b are configured to grip any of the tethers of the present invention, such as the tether 300 shown. This embodiment of the clamp 1000 includes a pair of skin retractors 1010a and 1010b, which serve to hold the skin around an incision away from the tether 300 for better user visibility and ease of manipulation.

FIG. 10C shows another view of a clamp 2000 of the present invention engaging a tether 300 of the present invention. The two clamp arms 1005a and 1005b are shown with skin retractors 1010a and 1010b and tether 300.

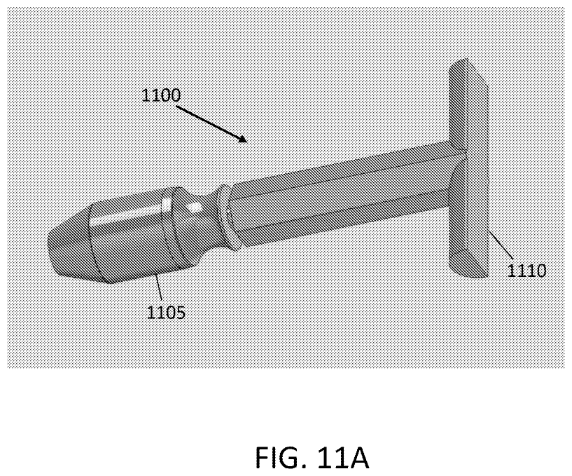

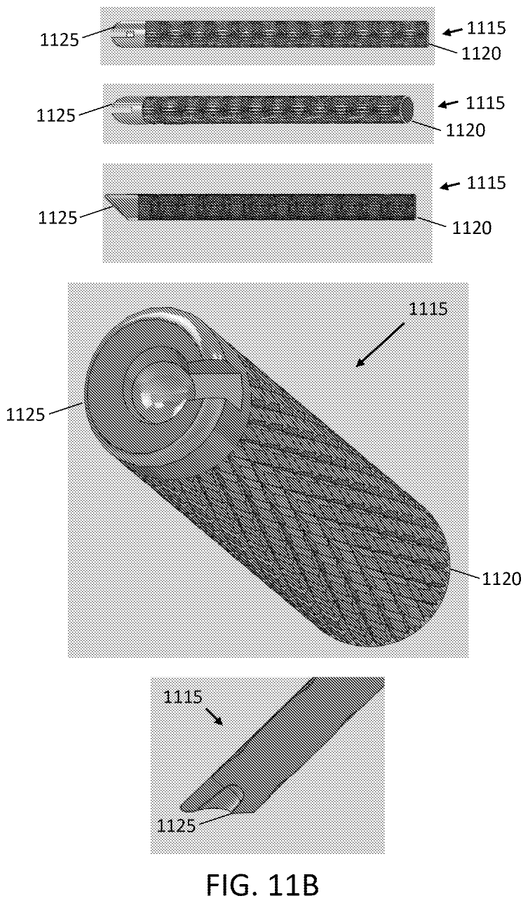

FIG. 11A shows an embodiment of a keyless chuck of the present invention and FIG. 11B shows an embodiment of an implantable bone device tamp of the present invention to be used in conjunction with the keyless chuck. The keyless chuck 1100 includes a receptacle 1105 and an impact anvil surface 1110. The keyless chuck 1100 may be used to tap an implantable bone device (not shown) with one end inserted into the receptacle 1105 and to turn an implantable bone device with one end inserted into the receptacle 1105. No key is required to secure the implantable bone device or the implantable bone device tamp in the receptacle of the keyless chuck. An embodiment of the keyless chuck of the present invention may also be used with an embodiment of the implantable bone device tamp of the present invention. FIG. 11B shows various views of the tamp 1115, the proximate end 1120 of which may be inserted into the receptacle 1105 and the distal end 1125 of which may be used to engage the implantable bone device (not shown) to tamp the implantable bone device into a bone at least to settle the implantable bone device in the bone such that the tether of the present invention can be used to secure the implantable bone device. One or more surfaces of embodiments of the keyless chuck and of the tamp of the present invention may be shaped (e.g., hexagonally, octagonally, etc.) or textured (e.g., knurled, roughened) to ease manipulation of the keyless chuck or the tamp.

A kit may include at least one of the tethers of the present invention; at least one of the targeting device of the present invention; at least one of the clamps of the present invention; at least one of the keyless chucks of the present invention; or at least one of the implantable bone device tamps of the present invention.

One skilled in the art will recognize that embodiments of the present invention, including tethers 100, 200, 300, and 400, the targeting device 700, and the clamps 1000 and 2000, the method embodiments 600, 800, and 900, and the keyless chuck 1100 and the implantable bone device 1115 provide small, implantable tethers for implantable bone devices that are capable of being fitted to a bone's contour, a targeting device for use with the intramedullary device tethers, clamps for use with the tethers, and a keyless chuck and an implantable bone device tamp for use with the tethers.

A tether for an implantable bone device comprises, consists essentially of, or consists of a retention portion for holding the implantable bone device in place, wherein the retention portion includes a recess configured to receive a proximate end of the implantable bone device when the implantable bone device is positioned inside a bone with the proximate end protruding from the bone, wherein the recess is open at a first end and closed at a second end; and a fastening portion for affixing the tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the retention portion.

A tether kit comprises, consists essentially of, or consists of a tether for an implantable bone device, the tether comprising, consisting essentially of, or consisting of, a retention portion for holding the implantable bone device in place, wherein the retention portion includes a recess configured to receive a proximate end of the implantable bone device when the implantable bone device is positioned inside a bone with the proximate end protruding from the bone, wherein the recess is open at a first end and closed at a second end; and a fastening portion for affixing the tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the nail portion.

A method for using tether for an implantable bone device comprises, consists of, or consists essentially of providing a patient requiring installation of the implantable bone device and the tether; installing the implantable bone device in a bone with a proximate end of the implantable bone device exposed; positioning the tether on the proximate end of the implantable bone device; and affixing the tether to the bone to hold the implantable bone device in place; wherein the tether includes a retention portion for holding the implantable bone device in place, wherein the retention portion includes a recess configured to receive a proximate end of the implantable bone device when the implantable bone device is positioned inside the bone with the proximate end protruding from the bone, wherein the recess is open at a first end and closed at a second end; and a fastening portion for affixing the tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the retention portion.

A computer-readable medium storing computer-readable instructions which, when acted upon by a 3D printer, cause the 3D printer to print a tether for an implantable bone device comprising, consisting of, or consisting essentially of a retention portion for holding the implantable bone device in place, wherein the retention portion includes a recess configured to receive a proximate end of the implantable bone device when the implantable bone device is positioned inside a bone with the proximate end protruding from the bone, wherein the recess is open at a first end and closed at a second end; and a fastening portion for affixing the implantable bone device tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the retention portion.

A targeting device for a bone drill or an implantable bone device comprises, consists of, or consists essentially of a handle; a sleeve attached to or integral with the handle at a proximal end of the sleeve, including at least a trough with a smoothly-curved cross-section and configured to receive a bone drill bit or the implantable bone device lengthwise; and at least one first spike at the distal end of the trough, configured to engage bone tissue to hold the distal end of the sleeve in place during use.

A method of using a targeting device for drilling bone comprises, consists of, or consists essentially of providing a patient in need of an implantable bone device installation through a hole drilled in a bone; providing the targeting device including: a handle; a sleeve attached to or integral with the handle at a proximal end of the sleeve, including at least a trough with a smoothly-curved cross-section and configured to receive a bone drill bit or the implantable bone device lengthwise; and at least one first spike at the distal end of the trough, configured to engage bone tissue to hold the distal end of the sleeve in place during use; making an incision over a drilling site; positioning the targeting device proximal to the drilling site, wherein the at least one first spike engages the bone proximal to the drilling site; aligning the tube to point to the drilling site; inserting a drill bit into the tube; and drilling the hole in the bone.

A method of using a targeting device for inserting an implantable bone device comprises, consists of, or consists essentially of providing a patient in need of an implantable bone device installation through a hole drilled in a bone; providing the targeting device including: a handle; a sleeve attached to or integral with the handle at a proximal end of the sleeve, including at least a trough with a smoothly-curved cross-section and configured to receive a bone drill bit or the implantable bone device lengthwise; and at least one first spike at the distal end of the trough, configured to engage bone tissue to hold the distal end of the sleeve in place during use; inserting the targeting device into an incision over a hole drilled in a bone; positioning the targeting device proximal to the hole, wherein the at least one first spike engages the bone proximal to the hole; aligning the trough, without the partial tube attached, to point to the hole; inserting the implantable bone device into at least a portion of the trough; and inserting the implantable bone device into the hole.

A clamp for manipulating a tether for an implantable bone device comprises, consists of, or consists essentially of two clamp arms joined at their proximal ends or between their proximal and distal ends and configured such that their distal ends can be brought together, wherein the distal ends of the two clamp arms are configured to grip the tether; wherein the tether comprises, consists of, or consists essentially of: a retention portion for holding the implantable bone device in place, wherein the retention portion includes a recess configured to receive a proximate end of the implantable bone device when the implantable bone device is positioned inside a bone with the proximate end protruding from the bone, wherein the recess is open at a first end and closed at a second end; and a fastening portion for affixing the tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the retention portion.

A keyless chuck is disclosed as comprises, consists essentially of, or consists of a receptacle configured to receive a proximal end of the implantable bone device or an implantable bone device tamp, wherein no key is required to secure the implantable bone device or the implantable bone device tamp in the receptacle, and wherein the receptacle is configured to rotate the implantable bone device or the implantable bone device tamp when the keyless chuck is rotated. In one aspect, the keyless chuck further includes an impact anvil surface. In another aspect, the keyless chuck further includes an implantable bone device tamp, wherein the tamp is detachably engageable with the receptacle.

A kit comprises, consists of, or consists essentially of at least one tether for an implantable bone device, each tether comprising, consisting or, or consisting essentially of a retention portion for holding an implantable bone device in place, wherein the retention portion includes a recess configured to receive a proximate end of the implantable bone device when the implantable bone device is positioned inside a bone with the proximate end protruding from the bone, wherein the recess is open at a first end and closed at a second end; and a fastening portion for affixing the tether to the bone, wherein the fastening portion includes one or more through fastener openings, wherein each through fastener opening is configured to receive a bone fastener, wherein the fastening portion is adjustable, and wherein the fastening portion is shaped to be less thick than a height of the retention portion; at least one targeting device, each targeting device comprising, consisting of, or consisting essentially of a handle; a sleeve attached to or integral with the handle at a proximal end of the sleeve, including at least a trough with a smoothly-curved cross-section and configured to receive a bone drill bit or the implantable bone device lengthwise; and at least one first spike at the distal end of the trough, configured to engage bone tissue to hold the distal end of the sleeve in place during use; at least one clamp, each clamp comprising, consisting of, or consisting essentially of two clamp arms joined at their proximal ends or between their proximal and distal ends and configured such that their distal ends can be brought together, wherein the distal ends of the two clamp arms are configured to grip the tether; and at least one keyless chuck, each keyless chuck comprising, consisting of, or consisting essentially of a receptacle configured to receive a proximal end of the implantable bone device or an implantable bone device tamp, wherein no key is required to secure the implantable bone device or the implantable bone device tamp in the receptacle, and wherein the receptacle is configured to rotate the implantable bone device or the implantable bone device tamp when the keyless chuck is rotated.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property(ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112(f) as it exists on the date of filing hereof unless the words “means for” or “step for” are explicitly used in the particular claim.

1. A tether for an implantable bone device, the tether comprising:
a retention portion for holding the implantable bone device in place, wherein the retention portion comprises a recess configured to receive a proximate end of the implantable bone device when the implantable bone device is positioned inside a bone with the proximate end protruding from the bone, wherein the recess is open at a first end and closed at a second end; and
a fastening portion for affixing the tether to the bone, wherein the fastening portion comprises one or more through fastener openings, wherein each through fastener opening is configured to receive a bone fastener, wherein the fastening portion is adjustable, and wherein the fastening portion is shaped to be less thick than a height of the retention portion.

2. The tether of claim 1, wherein the implantable bone device is an intramedullary rod, an intramedullary nail, an inter-locking screw, a Küntscher nail, a percutaneous screw, or a percutaneous pin.

3. The tether of claim 1, wherein the tether comprises a bone-side contour to approximate a contour of the bone to which it is to be affixed or is adjustable to create a bone-side contour to approximate a contour of the bone to which it is to be affixed.

4. The tether of claim 1, wherein the tether comprises a smooth outside contour to reduce irritation of tissue when implanted.

5. The tether of claim 1, wherein the fastening portion comprises a first tab disposed on a first side of the retention portion having a first through fastener opening and a second tab disposed on a second side of the retention portion having a second through fastener opening, or wherein the fastening portion comprises a single tab on one side of the retention portion having a single through fastener opening.

6. The tether of claim 1, further comprising one or more bone-side spikes protruding from a bone-side surface of the tether and configured to penetrate the bone when the tether is affixed to the bone.

7. A tether kit comprising:
a tether for an implantable bone device, the tether comprising:
a retention portion for holding the implantable bone device in place, wherein the retention portion comprises a recess configured to receive a proximate end of the implantable bone device when the implantable bone device is positioned inside a bone with the proximate end protruding from the bone, wherein the recess is open at a first end and closed at a second end; and
a fastening portion for affixing the tether to the bone, wherein the fastening portion comprises one or more through fastener openings, wherein each through fastener opening is configured to receive a bone fastener, wherein the fastening portion is adjustable, and wherein the fastening portion is shaped to be less thick than a height of the nail portion.

8. The tether kit of claim 7, further comprising one or more implantable bone devices.

9. The tether kit of claim 7, wherein the tether further comprises one or more bone-side spikes protruding from a bone-side surface of the tether and configured to penetrate the bone when the tether is affixed to the bone.

10. A method for using a tether for an implantable bone device comprising:
providing a patient requiring installation of the implantable bone device and the tether;
installing the implantable bone device in a bone with a proximate end of the implantable bone device exposed;
positioning the tether on the proximate end of the implantable bone device; and
affixing the tether to the bone to hold the implantable bone device in place;
wherein the tether comprises:
a retention portion for holding the implantable bone device in place, wherein the retention portion comprises a recess configured to receive a proximate end of the implantable bone device when the implantable bone device is positioned inside the bone with the proximate end protruding from the bone, wherein the recess is open at a first end and closed at a second end; and
a fastening portion for affixing the tether to the bone, wherein the fastening portion comprises one or more through fastener openings, wherein each through fastener opening is configured to receive a bone fastener, wherein the fastening portion is adjustable, and wherein the fastening portion is shaped to be less thick than a height of the retention portion.

11. (canceled)

12. The tether kit of claim 7, further comprising a targeting device for a bone drill or an implantable bone device, the targeting device comprising:
a handle;
a sleeve attached to or integral with the handle at a proximal end of the sleeve, comprising at least a trough with a smoothly-curved cross-section and configured to receive a bone drill bit or the implantable bone device lengthwise; and
at least one first spike at the distal end of the trough, configured to engage bone tissue to hold the distal end of the sleeve in place during use.

13. (canceled)

14. (canceled)

15. (canceled)

16. (canceled)

17. The method of claim 10, wherein the method of using a targeting device for drilling bone further comprises:
providing a patient in need of an implantable bone device installation through a hole drilled in a bone;
providing the targeting device comprising:
a handle;
a sleeve attached to or integral with the handle at a proximal end of the sleeve, comprising at least a trough with a smoothly-curved cross-section and configured to receive a bone drill bit or the implantable bone device lengthwise; and
at least one first spike at the distal end of the trough, configured to engage bone tissue to hold the distal end of the sleeve in place during use;
making an incision over a drilling site;
positioning the targeting device proximal to the drilling site, wherein the at least one first spike engages the bone proximal to the drilling site;
aligning the tube to point to the drilling site;
inserting a drill bit into the tube; and
drilling the hole in the bone.

18. The method of claim 10, further comprising:
removing the partial tube from the trough;
inserting the implantable bone device into at least a portion of the trough; and
inserting the implantable bone device into the hole.

19. The method of claim 10, wherein the method of using a targeting device for inserting an implantable bone device further comprises:
providing a patient in need of an implantable bone device installation through a hole drilled in a bone;
providing the targeting device comprising:
a handle;
a sleeve attached to or integral with the handle at a proximal end of the sleeve, comprising at least a trough with a smoothly-curved cross-section and configured to receive a bone drill bit or the implantable bone device lengthwise; and
at least one first spike at the distal end of the trough, configured to engage bone tissue to hold the distal end of the sleeve in place during use;
inserting the targeting device into an incision over a hole drilled in a bone;
positioning the targeting device proximal to the hole, wherein the at least one first spike engages the bone proximal to the hole;
aligning the trough, without the partial tube attached, to point to the hole;
inserting the implantable bone device into at least a portion of the trough; and
inserting the implantable bone device into the hole.

20. The tether of claim 1, further comprising clamp for manipulating a tether for an implantable bone device, the clamp comprising:
two clamp arms joined at their proximal ends or between their proximal and distal ends and configured such that their distal ends can be brought together, wherein the distal ends of the two clamp arms are configured to grip the tether; wherein the tether comprises:
a retention portion for holding the implantable bone device in place, wherein the retention portion comprises a recess configured to receive a proximate end of the implantable bone device when the implantable bone device is positioned inside a bone with the proximate end protruding from the bone, wherein the recess is open at a first end and closed at a second end; and
a fastening portion for affixing the tether to the bone, wherein the fastening portion comprises one or more through fastener openings, wherein each through fastener opening is configured to receive a bone fastener, wherein the fastening portion is adjustable, and wherein the fastening portion is shaped to be less thick than a height of the retention portion.

21. The tether of claim 12, wherein the distal ends of the two arms are configured to grip the fastening portion of the tether, wherein the two arms are joined rotationally and wherein one or both arms are capable of rotating to bring their distal ends together, wherein the two arms are joined at their proximal ends and wherein one or both arms are capable of flexing to bring their distal ends together, or wherein the distal ends of the two arms each comprise a skin retractor configured to retract skin around an incision from the tether when the distal ends of the two arms grip the tether.

22. The tether of claim 1, further comprising a keyless chuck for an implantable bone device, the keyless chuck comprising:
a receptacle configured to receive a proximal end of the implantable bone device or an implantable bone device tamp, wherein no key is required to secure the implantable bone device or the implantable bone device tamp in the receptacle, and wherein the receptacle is configured to rotate the implantable bone device or the implantable bone device tamp when the keyless chuck is rotated.

23. The tether of claim 22, further comprising an impact anvil surface.

24. The tether of claim 22, further comprising an implantable bone device tamp, wherein the tamp is detachably engageable with the receptacle.

25. The tether kit of claim 7, further comprising:
at least one tether for an implantable bone device, each at least one tether comprising:
a retention portion for holding the implantable bone device in place, wherein the retention portion comprises a recess configured to receive a proximate end of the implantable bone device when the implantable bone device is positioned inside a bone with the proximate end protruding from the bone, wherein the recess is open at a first end and closed at a second end; and
a fastening portion for affixing the tether to the bone, wherein the fastening portion comprises one or more through fastener openings, wherein each through fastener opening is configured to receive a bone fastener, and wherein the fastening portion is shaped to be less thick than a height of the retention portion;
at least one targeting device, each at least one targeting device comprising:
a handle;
a sleeve attached to or integral with the handle at a proximal end of the sleeve, comprising at least a trough with a smoothly-curved cross-section and configured to receive a bone drill bit or the implantable bone device lengthwise; and
at least one first spike at the distal end of the trough, configured to engage bone tissue to hold the distal end of the sleeve in place during use;
at least one clamp for manipulating the tether, each at least one clamp comprising:
two clamp arms joined at their proximal ends or between their proximal and distal ends and configured such that their distal ends can be brought together, wherein the distal ends of the two clamp arms are configured to grip the tether; and
at least one keyless chuck, each keyless chuck comprising:
a receptacle configured to receive a proximal end of the implantable bone device or an implantable bone device tamp, wherein no key is required to secure the implantable bone device or the implantable bone device tamp in the receptacle, and wherein the receptacle is configured to rotate the implantable bone device or the implantable bone device tamp when the keyless chuck is rotated.
